(19) United States
(12) Plant Patent Application Publication
(10) Pub. No.: US 2006/0230478 P1
(43) Pub. Date: Oct. 12, 2006

(54) Apple tree plant named 'ROHO 3615'

(50) MALUS DOMESTICA; APPLE

(76) Inventor: Hans Hofmann, Langensendelbach (DE)

Correspondence Address: THE WEBB LAW FIRM, P.C., 700 KOPPERS BUILDING, 436 SEVENTH AVENUE, PITTSBURGH, PA, 15219, US

(21) Appl. No.: 11/100,366

(22) Filed: Apr. 6, 2005

(51) Int. Cl. A01H 5/00

(52) U.S. Cl. PLT/161

(57) ABSTRACT

A new and distinct Malus domestica Borkh. plant that produces apples having an excellent sugar-acid balance and keeping quality, suitable for use as a table and dessert fruit.

### BOTANICAL CLASSIFICATION

Malus domestica Borkh.

### VARIETAL DENOMINATION

‘ROHO 3615’

### BACKGROUND OF THE INVENTION

The present invention comprises a new and distinct cultivar of apple tree botanically classified as Malus domestica Borkh. and known by the varietal name ‘ROHO 3615’. The new variety was discovered in 2000 in Langensendelbach, Germany. The new variety is the result of a stable fruit mutation of ‘Pinova’ (U.S. Plant Pat. No. 11,601). The new variety exhibits similar disease resistance, fruit storage capacity, tree growth, fruit quality, and fruit size to ‘Pinova’, but exhibits a full red color. The full red color of ‘ROHO 3615’, especially of the apples ripened in the shadow of the trees, distinguishes the new variety from other Malus domestica varieties.

The new variety has been trial and field tested and has been found to retain its distinctive characteristics and remain true to type through successive propagations.

### DESCRIPTION OF THE DRAWINGS

The accompanying photographic drawings illustrate the new cultivar, with the color being as nearly true as is possible with color illustrations of this type:

FIG. 1 shows the fruits of the new variety at maturity;

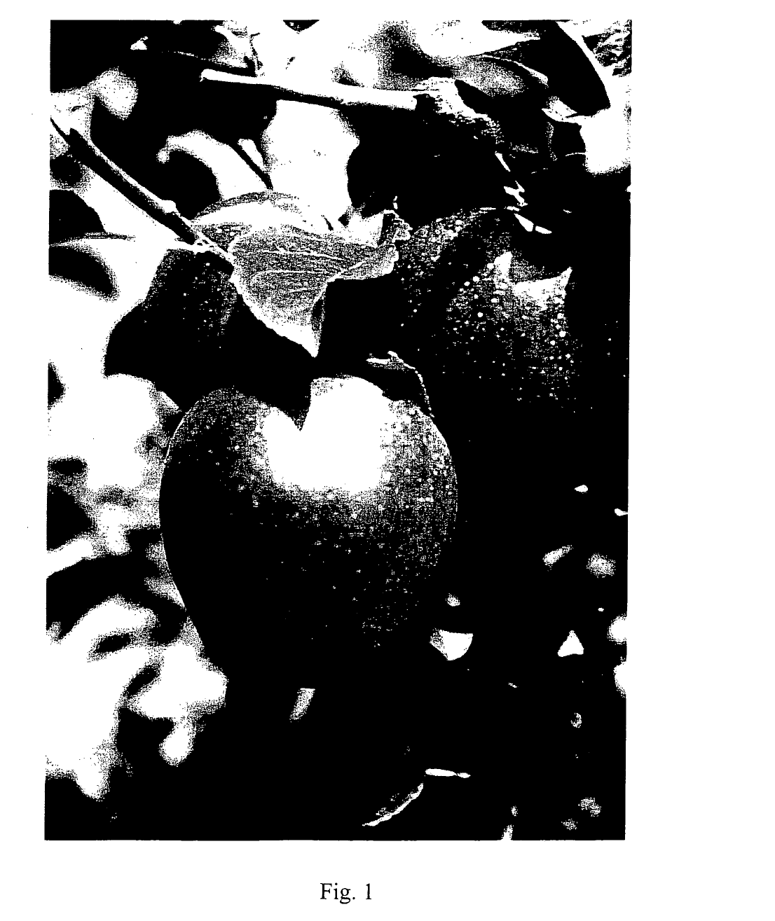

FIG. 2 shows the blooms of the new variety; and

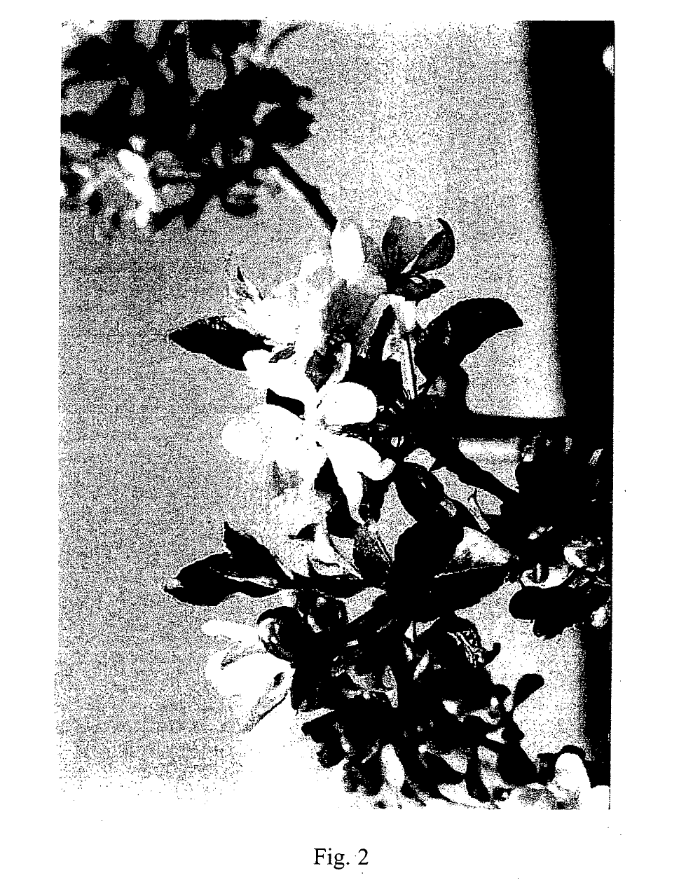

FIG. 3 shows an entire tree of the new variety.

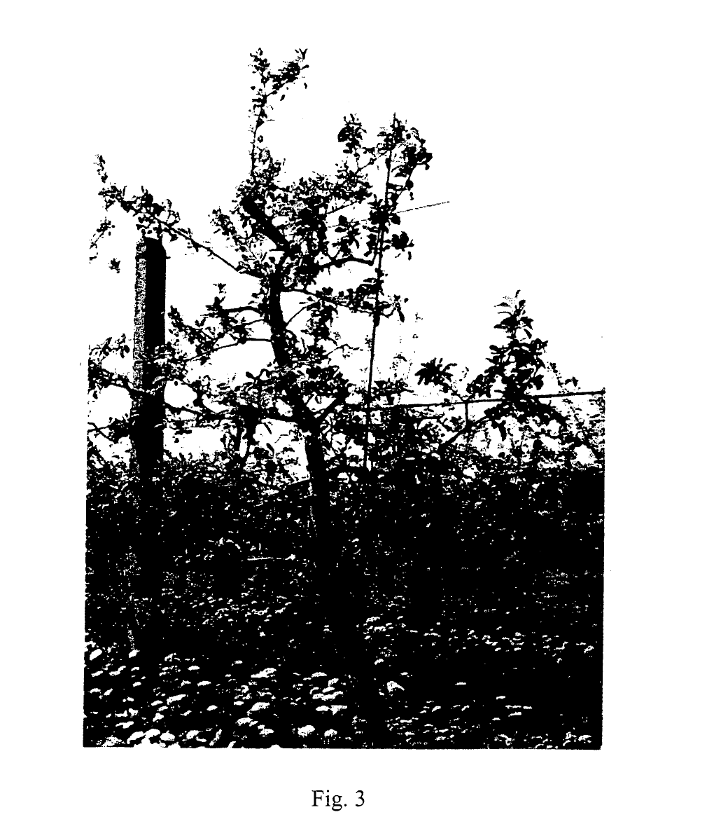

### DESCRIPTION OF THE PLANT

The following detailed description sets forth the characteristics of the new cultivar. The data which defines these characteristics was collected by asexual reproductions by budding and grafting first carried out in Langensendelbach, Germany in 2000. The new variety was grafted on M9 Rootstock and grown in a planting system field. The color readings were taken in natural daylight. Color references are primarily to the R.H.S. Colour Chart of The Royal Horticultural Society of London.

### Tree

Age: 3 years.
Size: 2.5 m high; small to medium in size.
Vigor: Dwarf.
Density: Not very dense with good ramification.
Form: Pyramidal.
Production: Very high and regular.
Growth type: Spindle bush; similar to ‘Pinova’.
Bearing: Regular with no alternate bearing.
Trunk:
   Size.—4.8 cm in diameter at 80 cm high at 3 years on M9 Rootstock.
   Surface texture.—Smooth.
   Bark color: 166B.
   Lenticels.—Length: Average 5.0 mm (from 4.0-8.0 mm). Width: 1.0 mm (from 0.9-1.9 mm). Color: Pale brown, between 164C and 165D. Density: High.
Branches:
   Diameter.—Normal to thin in size; at 1 year of age the diameter is 8.0 mm.
   Surface texture.—Smooth.
   Color.—1 year: 176A.
   Form.—Roundish in diameter with good ramification.
   Average angle.—60-75° in respect to the trunk.
   Bud arrangement.—Mutual. Internode: Average of 3.0 cm (from 2.5-5.0 cm).
   Lenticels.—Length: 2.0-3.5 mm. Width: 0.6-1.5 mm. Shape: Oblong to round. Density: Dense to very dense, similar to ‘Pinova’. Color: 164C to 164D.
Leaves:
   Length.—80.0-90.0 mm.
   Width.—50.0-65.0 mm.
Form: Ovate, like ‘Pinova’.
Base: Symmetric.
Apex: Tapering.
Margin: Crenulate.
Texture: Weak and wavy.
Thickness: Normal.
Pubescence:
   Upper surface.—Weak.
   Lower surface.—Weak to medium, younger leaves have more pubescence.
Color:
   Young leaves.—Upper surface: 144B. Lower surface: 144C to 145A.
   Mature leaves.—Upper surface: 143A. Lower surface: 143C.
Petiole:
   Shape.—Straight.
   Length.—2.5-3.8 cm.
   Diameter.—1.8-2.2 mm; measured at the middle of a 1 year old shoot.
   Color.—Between 141C and 143A.
Veins: Venation type: Net-like. Color: Upper surface: 145A. Lower surface: 147D.
Flowers:
   Buds.—Pedicels: Length: 2.5-3.5 cm. Diameter: 0.9-1.7 mm. Color: 133D to 138C.
   Bud (terminal).—Length: 5.0-8.0 mm. Width: 3.5-5.0 mm. Color: 178A.
Blooming time: Middle to late.
Blooming period: Long to very long (15 days in Germany).
Pollination requirements: Diploid; self-sterile; excellent pollinator.
Number of flowers per cluster: 5 to 9.
Fragrance: Mild.
Petals:
   Number.—5.
   Length.—21 mm.
   Width.—9.0-11.0 mm.
   Shape.—Oblong, tapering on the lower end.
   Margin.—Entire.
   Texture and appearance.—Solitary; rotate.
Color:
   When opening.—Upper surface: 56B. Lower surface: 57B-57C.
   When fully opened.—Upper surface: 56B and 155B. Lower surface: 57D and 155C.
Sepals:
   Shape.—Tapering.
   Margin.—Entire.
   Texture.—Rough.
   Length.—5.0-6.0 mm.
   Width.—1.3-1.8 mm.
   Color.—Upper surface: 144B. Lower surface: 144C.
   Stamens.—Number (per flower): 13-15. Filament length: 4.0-6.0 mm.
   Anthers.—Shape: Oblong. Length: 1.8-2.1 mm (average of 2.0 mm). Color: 3D.
   Pollen.—Color: 17A to 17C. Amount (generally): Heavy.
   Pistils.—Length: 7.0-10.0 mm; variable.
   Style.—Length: 6.0 mm. Color: 8C to 9D.
   Stigma.—Shape: Rounded. Color: 1B to 2B.
Fruit:
   Maturity when described.—Fully ripened.
   Date of picking.—The second week of October.
   Size.—Axial diameter: Average 69.0 mm (from 64.0 to 71.0 mm). Transverse diameter: Average 76.0 mm (from 70.0 to 80.0 mm).
   Form.—Conical.
   Cavity.—Shape: Flat. Depth: 7.0 mm. Breadth: 19.0 to 21.0 mm.
   Basin.—Shape: Medium to deep. Depth: 13.0 mm. Width: 26.0 mm.
   Calyx.—Closed.
Skin:
   Thickness.—Thin and firm.
   Texture.—Fine, with rare instances of minor russeting in the basin.
   Tendency to crack.—No cracking.
   Color.—46A to 46B.
   Ground Color.—151A, changing later to 12C to 13C.
Flesh:
   Aroma.—Mild.
   Color.—8D to 9D.
   Texture.—Firm; juicy; fine.
   Eating quality.—Excellent; well balanced sugar-acid content; sweet with a hint of sour; very good flavor.
Core:
   Bundle area.—Medium to wide; with a visible bundle.
   Capillary area.—Clear; medium to large.
   Calyx tube.—Closed.
   Styles.—8.0 mm.
   Axillary cavity.—Middle-deep to deep.
   Seed cells.—Wall: Firm. Depth: 12.0 mm. Breadth: 5.0-6.0 mm.
Seeds:
   Number perfect.—10 to 14 (very fertile).
   Number in one cell.—2-3.
   Length.—7.0-8.0 mm.
   Breadth.—2.5-3.0 mm.
   Form.—Oblong to ovoid.
   Color.—166A to 166B, up to 200D.
Stem:
   Length.—Average of 40.0 mm (range of 35.0-45.0 mm).
   Width.—Average of 1.4 mm.
   Color.—Initially 172B, changing to 199A.
Use: Table fruit; high quality dessert apple; long storage time.
Shipping quality: Excellent and firm, no bruising with normal handling.
Keeping quality: Excellent; apples last from 5 to 7 months on average; needs more than 90% (optimum 96%) humidity.
Tree winter hardiness: Hardy to −25° C. under weather conditions typical in Germany (better than ‘Jonagold’).
Bud winter hardiness: Hardy to −25° C. under weather conditions typical in Germany (better than ‘Elstar’).
Drought tolerance: Minimal, because the small fruits need irrigation under dry conditions.
Disease resistance: Low susceptibility to scab; medium susceptibility to mildew and fire blight; sometimes Gloeosporium rot occurs in storage.

I claim:

1. A new and distinct variety of Malus domestica Borkh. substantially as shown and described.
